(19) United States
(12) Patent Application Publication
(10) Pub. No.: US 2010/0077835 A1
(43) Pub. Date: Apr. 1, 2010

(54) PARTICULATE MATTER SENSOR

(76) Inventor: Matthew Brian Below, Findlay, OH (US)

Correspondence Address: HONEYWELL INTERNATIONAL INC.;PATENT SERVICES, 101 COLUMBIA ROAD, P O BOX 2245, MORRISTOWN, NJ, 07962-2245, US

(21) Appl. No.: 12/568,201

(22) Filed: Sep. 28, 2009

Related U.S. Application Data
(60) Provisional application No. 61/100,507, filed on Sep. 26, 2008.

(51) Int. Cl. G01N 33/00; G01R 3/00

(52) U.S. Cl. 73/23.31; 29/592.1

(57) ABSTRACT

Exemplary embodiments of the present invention relate monitoring particulate matter within an exhaust gas stream. In one exemplary embodiment, a particulate matter sensor for an exhaust system of an engine is provided. The sensor includes a housing having a first end and a second end. The housing includes a sealing feature located at the first end of the housing. The housing further includes an attachment feature located between the first end and the second end of the housing. The sensor further includes a sensing rod extending from the first end of the housing. The sensing rod is configured to generate a signals based upon particulate matter flowing within the exhaust system of the engine. The sensor also includes an electrical connector extending from the second end of the housing. The electric connector is in communication with the sensing rod to transmit signals generated by the sensing rod.

### CROSS REFERENCE TO RELATED APPLICATION

The present application claims the benefit of U.S. Provisional Patent Application Ser. No. 61/100,507 filed Sep. 26, 2008, the contents of which is incorporated herein by reference thereto.

### FIELD OF THE INVENTION

Exemplary embodiments of the present invention relate to methods and devices for monitoring the flow of particulate matter within an exhaust gas stream. More specifically, in one exemplary embodiment, the present invention relates to diesel particulate sensors having improved sensing capabilities

### BACKGROUND

Emissions from stationary and mobile fossil burning devices have been and will continue to be of particular concern in view of accumulating laws and regulations restricting emissions from such devices. In one aspect, particulate matter within emissions has been regulated causing industries, particularly the automotive industry, to utilize particulate matter removal devices, such as filters. Such removal devices are configured to catch or trap particulate matter flowing through an exhaust gas stream prior to exiting an exhaust system. To determine when the particulate matter filter is reaching its capacity, the total volume or volume flow rate of particulate matter flowing into the filter or within the exhaust gas stream is monitored. Monitoring is often achieved through a particulate matter sensor exposed to the exhaust gas flow. The particulate matter sensor functions by transmitting signals based upon electrical potential across the probe of the sensor. For example, as ionized particulate matter within the exhaust gas passes across the sensor, electrical potential across the sensor increases or decreased providing an indication of the amount of particulate matter that has traveled past the sensor and into the filter.

However, many of these sensors fail to provide accurate readings of particulate matter flowing past the sensor or within an exhaust gas stream. For example, certain sensors are not sufficiently robust to withstand forces or temperatures encountered by such sensors. Other problems with certain sensors are their inability to accurately indicate the presence of particulate matter within an exhaust gas flow due to poor signal noise ratio. Still other problems exist as well. Accordingly, in view of the shortcomings of previous sensor designs, there is a need for improved methods and devices for monitoring the flow of particulate matter flowing within and exhaust gas stream.

### SUMMARY OF THE INVENTION

Exemplary embodiments of the present invention relate to methods and devices for monitoring the flow of particulate matter within an exhaust gas stream. In one exemplary embodiment, a particulate matter sensor for an exhaust system of an engine is provided. The particulate matter sensor includes a housing having a first end and a second end. The housing includes a sealing feature located at the first end of the housing for forming a seal between the housing and a corresponding component. The housing further includes an attachment feature located between the first end and the second end of the housing for attachment of the particulate matter sensor to the exhaust system. The particulate matter sensor further includes a sensing rod extending from the first end of the housing. The sensing rod is configured to generate a signals based upon particulate matter flowing within the exhaust system of the engine. The particulate matter sensor also includes an electrical connector extending from the second end of the housing. The electric connector is in communication with the sensing rod to transmit signals generated by the sensing rod.

In another exemplary embodiment, a method of monitoring particulate matter flowing within an exhaust gas stream of an engine is provided. The method includes: forming an opening through an exhaust conduit of an exhaust system, the opening defining a first sloped surface and a first threaded portion; forming a particulate matter sensor having a housing, sensing rod and electrical connector, the housing including a first end and a second end, the housing further including a second sloped surface disposed at the first end and a second threaded portion located between the first end and the second end; and threadably engaging the first and second threaded portions to cause engagement and sealing of the first and second sloped surface, wherein upon engagement the sensing rod is located within an exhaust gas stream and generates signals based upon the presence of particulate matter, the signals being transmitted to a controller through the electrical connector.

The above features and advantages and other features and advantages of the present invention are readily apparent from the following detailed description of the best modes for carrying out the invention when taken in connection with the accompanying drawings.

### BRIEF DESCRIPTION OF THE DRAWINGS

Other objects, features, advantages and details appear, by way of example only, in the following detailed description of embodiments, the detailed description referring to the drawings in which:

FIG. 1 illustrates a schematic view of an emission control system including one or more particulate matter sensor assemblies according to exemplary embodiments of the present invention;

FIG. 2 illustrates a cross-sectional view of an exemplary embodiment of a particulate matter sensor assembly according to the teachings of the present invention; and

FIG. 3 illustrates an enlarged view of the particulate matter sensor assembly shown in FIG. 2.

### DETAILED DESCRIPTION OF EXEMPLARY EMBODIMENTS

Exemplary embodiments of the present invention provide methods, systems and devices for detecting and monitoring particulate matter flowing in an exhaust gas stream. In one particular exemplary embodiment, a particulate matter sensor is provided. The particulate matter sensor is configured for detecting and monitoring particulate matter flowing within an exhaust gas stream for determining volume or volume flow rate of particulate matter flowing within the stream. In one particular exemplary embodiment, the particulate matter sensor includes a sensing rod having an increased surface area for improving accuracy in the detection and monitoring of particulate matter within the exhaust gas stream. In one configuration, the increased surface area of the sensor rod is achieved through the structure of the particulate matter sensor, which allows for larger diameter sensing rods to be used. Other advantageous will become apparent as shown and described herein.

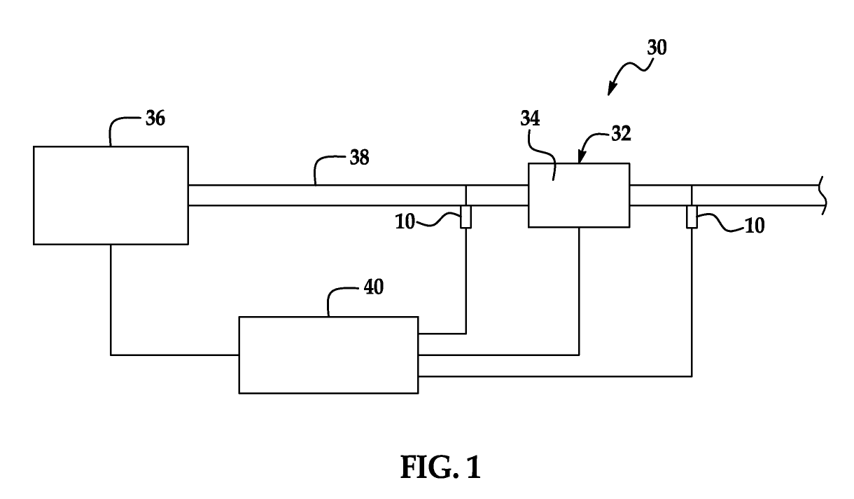

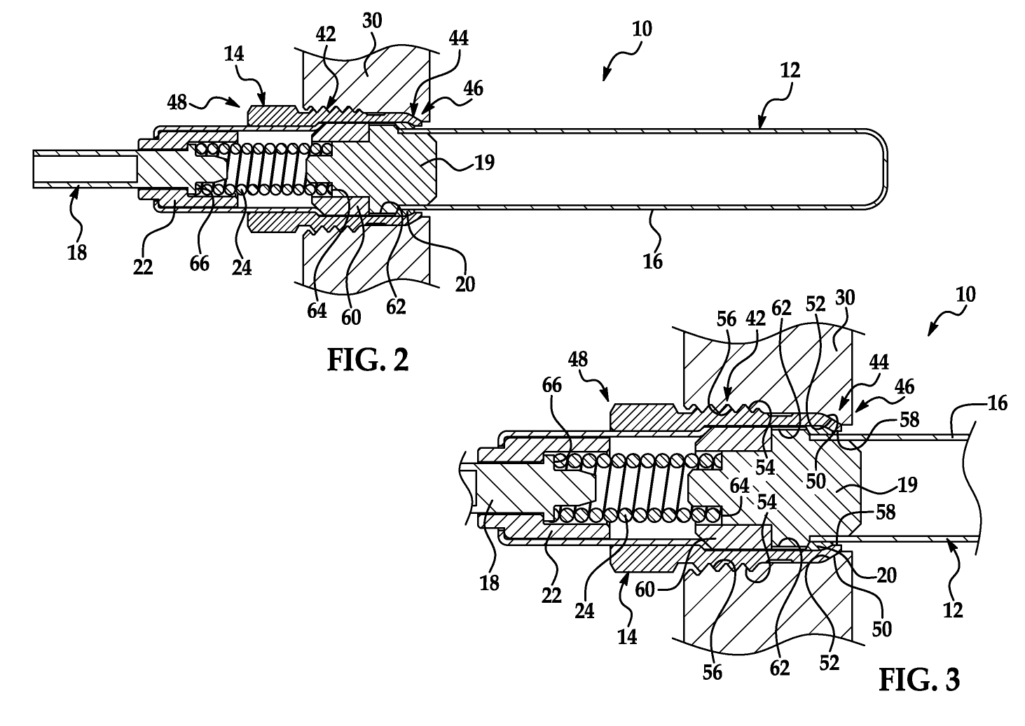

In general, referring to FIG. 2, an exemplary embodiment of an improved particulate matter sensor assembly 10 is provided. The sensor assembly includes a sensing rod 12 supported by a housing 14. The sensing rod includes a probe 16 configured for placement within an exhaust gas stream for detecting particulate matter flowing within the stream. In one exemplary embodiment, the sensing rod 12 and/or probe comprises a hollow tube member. The sensor assembly 10 further includes an electrical connector 18 for providing communication between the sensing rod 12 and a controller 40 (see FIG. 1). The sensing rod 12 also includes a mount 19 for attachment of the sensing rod to the housing 14 and providing electrical communication between the sensing rod 12 and the electrical connector 18. The sensor assembly further includes a first insulator 20, second insulator 22 and a third insulator 60. In one embodiment, the first insulator 20 comprises a compressed talc powder or any other equivalent dielectric material for electrically insulating the exterior surface of the end of sensing rod from a securement feature 44 of the housing. In addition, the second insulator 22 comprises a ceramic material for electrically insulating the electrical connector 18 from the housing 14 and the third insulator also comprises a ceramic material for electrically insulating mount 19 from housing 14 as well. In one configuration, the sensor assembly 10 includes an intermediate connector 24 (such as a resilient coil spring member) for providing communication between the electrical connector 18 and the sensing rod 12, via mount 19.

In one exemplary operation, referring to FIG. 1, an exhaust control system 30 is providing for monitoring and removing particulate matter from an exhaust gas stream. The exhaust control system 30 includes and exhaust control device 32, such as a diesel particulate filter 34, in fluid communication with an engine 36 through a suitable exhaust gas conduit 38. The exhaust control system 30 also includes one or more particulate matter sensor assemblies 10 located before and/or after the exhaust control device. In one exemplary embodiment, as exhaust gas flows through the exhaust gas conduit 38 the total volume of particulate matter that flows past the sensor assembly 10, and eventually into an exhaust control device 32, for a given time period is determined by monitoring the electrical potential, or change in electrical potential, formed along a surface of the sensing rod 12 and more particularly the probe 16. Signals, based upon this electrical potential are generated and transmitted to a suitable receiver. In one configuration, as exhaust gas travels past probe 16, a negative or positive potential is accumulated across the surface of the probe. This potential is related to the total amount of exhaust gas which has passed the probe 16 as negative or positive particles from the exhaust gas gravitate towards the probe. As such, by generating signal based upon the amount of electric potential or change in electrical potential it is possible to determine the amount of particulate matter that has flowed by the sensor assembly 10 and hence into the exhaust control device 32.

Reference is also made to the following patent applications: U.S. Provisional Patent Application Ser. No. 61/083,328 filed Jul. 24, 2008 and U.S. patent application Ser. No. 12/508,272, filed Jul. 23, 2009; U.S. patent application Ser. No. 12/467,673, filed May 18, 2009; and U.S. Provisional Patent Application Ser. No. 61/083,333 filed Jul. 24, 2008 and U.S. patent application Ser. No. 12/508,096 filed Jul. 23, 2009, the contents of each of the aforementioned provisional and non-provisional applications are incorporated herein by reference thereto.

Reference is also made to the following U.S. Pat. Nos. 6,971,258; 7,275,415; 5,697,334 and 4,111,778 the contents each of which are incorporated herein by reference thereto.

In one exemplary embodiment, a controller 40 is provided for receiving the signals and determining the total amount of particulate matter that has flowed past the sensing rod 12 and hence into the exhaust control device 32. In this embodiment, the controller includes suitable algorithms for determining the amount of particulate matter flowing past the sensor assembly 10 and hence into the exhaust control device 32. In one exemplary embodiment, the controller may also be in communication with one or more regeneration devices or configurations, for causing an increase in temperature of the exhaust control device 32 and/or sensor assembly 10 suitable for causing removal or inhalation of accumulated particles. As such, once the controller determines that total volume of particulate matter that has passed the sensor assembly 10 has reached a predetermined level, the controller 40 may initiate regeneration of the exhaust control device 32 and the particulate matter sensor assembly 10.

In greater detail, referring to both FIGS. 1 and 2, the housing 14 is configured to provide mounting of the particulate matter sensor assembly 10 to a component of an exhaust control system 30 such as conduit 38, exhaust control device 32 or otherwise. Accordingly, the particulate matter sensor assembly 10 includes an attachment feature 42 and a sealing feature 44 for engagement and sealing of the particulate matter sensor assembly 10 to the component of the exhaust control system 30. In one exemplary embodiment, referring to FIG. 3, the particulate matter sensor assembly 10 includes a housing 14 having a first end 46 and a second end 48. The first end 46 of the housing 46 includes sealing feature 44 for engagement with a corresponding component of the exhaust control system 30, such as an exhaust conduit, exhaust treatment device or otherwise. In the configuration shown, the sealing feature comprises an annular ring having a sloped surface 50 for engagement with a corresponding sloped surface 52 of the exhaust control system 30. Located between the first end 46 and the second end 48 of the housing 14 is disposed the attachment feature 42. In the configuration shown, the attachment feature 42 is disposed approximately half way between the first end 46 and the second end 48 of the housing 14. The attachment feature may comprise any suitable attachment feature. However, in one exemplary embodiment the attachment feature 42 comprises a threaded portion 54 adapted for engagement with corresponding threaded portion 56 of the exhaust control system 30. In this exemplary embodiment, during engagement, e.g., rotation of the threaded portion 54 of the housing with respect the threaded portion 56 of the exhaust control system, the sloped surface 50 of the housing 14 engages the sloped surface of the exhaust control surface thereby forming a seal therebetween.

The above referenced configuration is particularly advantageous as it allows for increased diameter of the sensing rod 12. This is due to the placement of the sealing feature at an end portion (e.g., first end 46) of the housing 14. In prior configurations, the sealing feature was located on a side of the attachment feature that is opposite of the sensing rod, e.g., between the first and second end of the housing. As such, the attachment feature, e.g., threads, are formed within the diameter of the sealing feature, which results in a smaller diameter sensing rod. Through the configuration of the housing of the present invention, larger sensing rods 12 can be used, which provide for increased sensing accuracy and improves robustness of the sensing rod due to the larger mounting diameter. This is because by placing the sealing feature 44 at an end of the housing 14, the use of a sealing surface extending beyond an attachment feature, such as done in prior sensor assemblies, is no longer necessary. This allows for the diameter of the probe to be more similar to the diameter of the attachment feature, or threads, thereby allowing for a larger probe.

For example, previous sensors have been limited to 4 mm probes with 14 mm threads or 6.5 mm probes with 18 mm threads. Through the features of the present invention, it is possible to utilize 10 mm probes with 16 mm threads. Not only does this provide for increased accuracy of the particulate matter sensor, but also provides for a more robust sensor assembly 10 due to the larger mounting base of the probe 16, for mounting to mount 19. It should be appreciated that other larger diameter probes and threads are possible. Accordingly and in one exemplary embodiment, the outer diameter of the sensing rod is at least about 50% of a diameter of the sealing feature of the housing. In another embodiment, the outer diameter of the sensing rod is at least 85% of a diameter of the sealing feature of the housing. Of course, other suitable diameters greater or less than the aforementioned values are considered to be within the scope of exemplary embodiments of the present invention.

Still referring to FIG. 3, the sensing rod is mounted to the mount 19 through a suitable attachment means. In one exemplary embodiment, the sensing rod 12 is configured to engage the mount 19 and prevent or substantially limit rotation of the sensing rod with respect to the mount. In one configuration, the sensor assembly includes a high temperature resistant electrically conductive adhesive for bonding of the sensing rod 12 to the mount 19. Examples of suitable high temperature adhesives include silver epoxy or equivalents thereof. In another configuration the sensing rod 12 is welded, such as laser welded, brazed or any other equivalent process as long as there is an electrically conductive securement of the rod to the mount. Other configurations are possible.

To prevent grounding of the sensing rod 12, the first insulator 20 comprising a compressed powder dielectric material (e.g., talc or equivalent thereof) is disposed between the sensing rod and the housing 14. The configuration of the first insulator and housing 14 also forms a gap 58 between the sensing rod 12 and the housing. Alternatively, gap 58 may be filled with the material of the first insulator 20. In one exemplary embodiment and through the configuration and placement of the first and third insulators, an air gap 62 is also formed between the mount 19, which is electrically conductive, and the housing 14. As such, through this configuration signals formed by the sensing rod 12 are prevented from grounding through the housing 14.

The sensing rod 12 may be formed of any suitable material for detection of particulate matter or other material of interest. In one configuration, the sensing rod is formed of an electrically conductive or semi-conductive material and is also capable of withstanding deleterious effects of exhaust emission (e.g., heat, corrosiveness, or otherwise). For example, the sensing rod may be formed of conducting or semiconducting material such as metal, metal alloy or otherwise. In another example, the sensing rod may be formed of an insulating material, such as ceramic or glass, and include a conductive or semi-conductive layer thereover, such as metal, metal alloy or otherwise. In either of these configurations, or otherwise, the conductive or semi-conductive material may include a non-conducting or dielectric layer applied or placed thereover for protection of the conductive material or otherwise, such as described below. Examples of some suitable conducting materials include nickel alloys such as HAYNES® 214® or HAYNES® 240®, both of which are sold by Haynes International Inc. of Kokomo, Ind., U.S.A.

Similarly, the sensing rod 12 may be formed through any suitable forming process including molding, stamping or otherwise. In one exemplary embodiment, the probe 16 of the sensing rod comprises a hollow tube member. In this configuration, it is contemplated that the probe 16 and/or sensing rod 12 is formed through a deep drawn process, or the like. Other methods are possible.

In one exemplary embodiment, the sensing rod 12 is formed with, generates or otherwise includes an insulating material or layer thereover to prevent transmission of electric current to or from unwanted components. In one configuration, the insulating material or layer comprises an oxide coating of a material forming the sensing rod. Suitable materials include materials capable of forming an oxide coating or layer that has low thermal and/or electrical conductivity. One exemplary material includes a first material comprising nickel alloy and a second material comprising aluminum and one or more of yttrium or zirconium. Other materials and combinations are possible.

As previously mentioned, the sensing rod 12 is in communication with one or more additional devices for transmission of signals from the probe 16 to another device, through electrical connector 18. In one exemplary embodiment, the sensing rod 12 is attached directly to the electrical connector 18. In another exemplary embodiment, referring to FIG. 3, the sensing rod 12 is attached to the electric connector 18 through a resilient intermediate connector 28, such as a coil spring or otherwise. In this configuration, the mount 19 includes a first collar 64 for providing reactionary force against the resilient intermediate connector. Similarly, the electrical connector 18 also includes a second collar 66 for providing reactionary force against the resilient intermediate connector. Other configurations are possible.

In one exemplary embodiment, referring to FIG. 1, the sensor assembly is in communication with controller 40. In one configuration, the controller 40 comprises a controller for an exhaust control system 30. In another exemplary embodiment, the controller 40 is part of an electronic control unit of a vehicle. In either of these configurations, the control unit is configured to transmit and receive signals from the particulate matter sensor assembly 10. Such information is particularly advantageous for determining the amount of particulate matter that has flowed through the exhaust control device 32 in a given time period or cycle interval such as between regenerations of the exhaust control device. Accordingly, the controller 40 may cause regeneration of the exhaust control unit and/or sensor based upon the particulate matter flowing within the exhaust gas as indicated by the particulate matter sensor assembly 10.

The particulate matter sensor assembly 10 may be used in various industries for determining a flow of particulate matter. These industries include, without limitation, automotive industry, freight industry, mass transit industry, power generating industry such as power plants or factors, or other emission producing industry. In one particularly advantageous application, the particulate matter sensor assembly 10 is useable within the automotive industry and more particularly with internal combustion engines of vehicles for monitoring particulate matter generated thereby. In this configuration, the particulate matter sensor assembly 10 is placed within the exhaust gas stream flowing through an exhaust gas conduit, exhaust treatment device or otherwise, from a diesel engine, gasoline engine or otherwise.

In one exemplary embodiment, an exhaust control system is provided for monitoring and removing particulate matter from an exhaust gas stream. The exhaust system includes and exhaust control device, such as a particulate matter filter, which is in fluid communication with an engine through a suitable exhaust gas conduit. The exhaust control system also includes one or more particulate matter sensors. As exhaust gas flows through the exhaust gas conduit, particulate matter for a given time period is determined by monitoring an electrical signal across a surface of the probe generated by an electrical potential of particles flowing past the probe to determine the amount of particulate matter that has flowed into the exhaust control device. The particulate matter sensor generates signals based upon the charged particles flowing past the probe. The signals are received by a controller configured for determining the total amount of particulate matter that has flowed past the probe and into the particulate matter filter based upon the signals received.

Further exemplary embodiments include monitoring particulate matter flowing within an exhaust gas stream using a sensing rod constructed in accordance with exemplary embodiments of the present invention. In one embodiment, the method includes generating signals with the particulate matter sensor based upon the presence of particulate matter flowing in the exhaust gas stream and flowing past the sensor and thus creating an electrical signal in the probe based upon the electrically charged particles or the electrical potential of the particles flowing past the sensing rod of the probe. As previously mentioned and in one exemplary embodiment, the signal is based upon a charge created in the probe based upon particulate matter flowing past the sensor. The controller receives the signals and determines at least one flow characteristic of particulate matter flowing within the exhaust gas stream such as total amount of particulate matter flowing by the sensor and into the emission control device, or volume flow rate of particulate matter or otherwise.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiments disclosed as the best mode contemplated for carrying out this invention, but that the invention will include all embodiments falling within the scope of the present application.

What is claimed is:

1. A particulate matter sensor for an exhaust system of an engine, comprising:
a housing having a first end and a second end, the housing including a sealing feature located at the first end of the housing for forming a seal between the housing and a corresponding component, the housing further including an attachment feature located between the first end and the second end of the housing for attachment of the particulate matter sensor to the exhaust system;
a sensing rod extending from the first end of the housing, the sensing rod being configured to generate a signals based upon particulate matter flowing within the exhaust system of the engine; and
an electrical connector extending from the second end of the housing, the electric connector being in communication with the sensing rod to transmit signals generated by the sensing rod.

2. The particulate matter sensor of claim 1, wherein the sealing feature includes a diameter that is less than a diameter of the attachment feature.

3. The particulate matter sensor of claim 2, wherein the sealing feature comprise an annular ring.

4. The particulate matter sensor of claim 3, wherein the annular ring includes a sloped surface having a decreasing diameter in a direction away from the housing, the sloped surface being configured to engage a corresponding sloped surface of the exhaust system.

5. The particulate matter sensor of claim 1, wherein the attachment feature comprises a threaded portion that is located generally half way between the first end and the second end of the housing, the threaded portion being configured to engage a corresponding threaded portion of an exhaust system to cause engagement between the sealing feature and the exhaust system.

6. The particulate matter sensor of claim 1, wherein the sensing rod includes an outer diameter that is at least about 85% of a diameter of the sealing feature of the housing.

7. The particulate matter sensor of claim 6, wherein the sensing rod comprises a hollow tube member.

8. The particulate matter sensor of claim 7, further comprising a mount located at the first end of the housing for engaging an opening formed by the hollow tube member.

9. The particulate matter sensor of claim 1, wherein the sensing rod is formed of a substantially non-conductive base member having an electrically conducting layer thereover and a non-conductive layer applied over the electrically conducting layer.

10. The particulate matter sensor of claim 1, wherein the sensing rod is formed of an electrically conducting base member having a non-electrically conducting layer applied thereover.

11. The particulate matter sensor of claim 1, wherein the sensing rod includes an outer diameter that is at least about 50%.

12. A method of making a particulate matter sensor for mounting to an exhaust treatment system, comprising:
forming a threaded engagement feature on an exterior surface of a housing portion of the particulate matter sensor, the threaded engagement feature being disposed between a first end of the housing portion and a second end of the housing portion, wherein a sealing feature is disposed closer towards the first end of the housing portion than the second end of the housing portion and is proximate to a sending rod of the particulate matter sensor.

13. The method of claim 12, wherein the sealing feature comprise a first sloped surface adapted to engage a second sloped surface of a corresponding component of the exhaust treatment system, the sloped surface having a decreasing diameter in a direction away from the housing portion.

14. The method of claim 12, wherein the sensing rod includes an outer diameter that is at least about 85% of a diameter of the sealing feature of the housing.

15. The method of claim 14, wherein the sensing rod comprises a hollow tube member.

16. The method of claim 15, further comprising a first insulator member located at the first end of the housing portion for engaging an opening formed by the hollow tube member and a second insulator member located at the second end of the housing portion configured to engage an electrical connector, the first and second insulator members being located within and supported by the housing portion.

17. The method of claim 12, wherein the sensing rod is formed of a substantially non-conductive base member having an electrically conducting layer thereover and a non-conductive layer applied over the electrically conducting layer.

18. The method of claim 12, wherein the sensing rod is formed of an electrically conducting base member having a non-conductive layer applied thereover.

19. The method of claim 12, wherein the sensing rod includes an outer diameter that is at least about 50% of a diameter of the sealing feature of the housing.
